(19) United States
(12) Patent Application Publication
(10) Pub. No.: US 2021/0371820 A1
(43) Pub. Date: Dec. 2, 2021

(54) METHOD FOR AMPLIFYING ANTIGEN-SPECIFIC REGULATORY T CELLS IN VITRO

(71) Applicant: Nanjing Airui Biological Technology Co., LTD, Nanjing, Jiangsu (CN)

(72) Inventors: Ling Lv, Nanjing, Jiangsu (CN); Jian Gu, Nanjing, Jiangsu (CN)

(21) Appl. No.: 16/342,193

(22) PCT Filed: Feb. 8, 2018
(86) PCT No.: PCT/CN2018/075730

§ 371 Date: Apr. 15, 2019

(30) Foreign Application Priority Data
Dec. 1, 2017 (CN) 201711252131.9

(51) Int. Cl. C12N 5/0783

(52) U.S. Cl. CPC C12N 5/0637; C12N 2501/15; C12N 2501/2302; C12N 2502/1121; C12N 2501/2315; C12N 2501/22; C12N 2501/2304

(57) ABSTRACT

The invention discloses a method for amplifying antigen-specific regulatory T cells in vitro, belonging to the technical field of biomedicine. The invention adopts Rapamycin combined with TGF-β cells to induce human T cells into antigen-specific regulatory T cells with immunosuppressive function in vitro by the action of DC cells, which has the advantages as follows: 1) the amount is sufficient; 2) the obtained regulatory T cells can resist differentiation to Th17 cells; 3) the obtained regulatory T cells have stronger inhibitory functions and biological effects compared with the regulatory T cells induced by other methods. The invention overcomes many defects of natural regulatory T cells and has greater therapeutic advantages in the treatment of inflammatory diseases, autoimmune diseases and prevention of organ transplant immunological rejection.

### FIELD OF THE INVENTION

The invention relates to a method for amplifying antigen-specific regulatory T cells in vitro, belonging to the technical field of biomedicine.

### DESCRIPTION OF THE RELATED ART

For half a century, immunosuppressive drugs significantly prolong the survival time of allografts, and also cause a series of serious problems such as clinical complications due to their own toxic effects, expensive price and drug-induced non-specific immunosuppression, which has greatly limited the further development of clinical organ transplantation. It is one of the basic subjects in the field of transplantation immunology by inducing graft-specific immune tolerance or achieving a “clinical almost tolerated” state similar to the application of immunosuppressive agents, thereby reducing or even eliminating immunosuppressive agents. Although many researchers have successfully induced immune tolerance in allogeneic transplants in different animals, however, due to some unclear reasons, an effective method for amplifying regulatory T cells has not been found so far, in which these experimental results can be repeated on a large pre-clinical animal model, let alone in clinical application. Therefore, the development of effective therapeutic strategies for inducing donor-specific immune tolerance has become the main direction of experimental and clinical research, which is characterized not only by prolonging the survival rate of grafts, but also eliminating the disadvantage of non-specific inhibition of immunosuppressive agents. Scholars at home and abroad have been seeking ways to replace immunosuppressive drugs, for example: (1) block of the costimulatory pathway: although the survival time of allografts is significantly prolonged by blocking the costimulatory pathway in the transplanted animal model, this method does not achieve sustained stable tolerance; (2) immature allogeneic dendritic cells (DC): the induced immature DC cells in vitro in some cases induce no response to antigen-specific T cells in vitro and in vivo, and the infusion in vivo can significantly prolong the survival of allografts, but only short-term tolerance can be obtained; (3) mixed chimerism: a large number of experimental studies indicate that mixed chimeric induction of immune tolerance is very close to clinical application, but a key issue is donor cell infusion, which may induce graft versus host disease (GVHD). The above measures have not achieved satisfactory results. At the same time, studies have shown that the mechanism by which these pathways induce immune tolerance is associated with secondary induction of CD4+CD25+ regulatory T cells (Treg).

In 1995, Sakaguchi et al. found that the deprivation of CD4+CD25+ regulatory T cells of mice caused a variety of autoimmune diseases, and the reinfusion of these regulatory T cells inhibited the disease. There are about 5% to 10% of cells continuous high expression CD25 molecule (IL-2 receptor alpha chain) in the CD4+ T cells of peripheral blood and lymphoid tissues of normal humans and mice, and at the same time, the subpopulation of cells is lowly expressed by the CD45RB molecule. Regulatory T cells subpopulation are derived from the thymus or from peripherally activated cells, and their T cell receptors (TCR) expression differs from conventional T cells in that they react with specific antigens in the periphery along with conventional CD4+ T cells. The thymus-derived CD4+CD25+ regulatory T cells need to be influenced by various factors such as stimulation of peripheral autoantigens to become functional regulatory T cells. Thymic epithelial cells may play a major role in the differentiation of CD4+CD25+ regulatory T cells, and the maturation or activation of antigen-presenting cells (APCs), particularly dendritic cells (DCs), performs the regulation function of CD4+CD25+ regulatory T cells. The transcription cytokine Foxp3 is a characteristic marker of the regulatory T cells, and is a specific transcription cytokine for the regulation and function of regulatory T cells; CD28 and its signaling pathways, IL-2 and IL-2 receptor signaling pathways are all necessary signal molecules for their survival. The transduction of the Foxp3 gene into both CD4+ and CD8+ cells enables them to be transformed into regulatory T cells. CD4+CD25+ regulatory T cells have been shown to have acute GVHD that controls type I diabetes and prevents hematopoietic stem cells; its potential protection against grafts has been demonstrated in allogeneic organ transplantation. However, current applications for regulatory T cells are still limited by the following conditions: 1) the number of naturally-produced regulatory T cells (natural Tregs) is small. Natural Tregs account for about 5% to 10% of thymus and peripheral blood CD4+ in animals, whereas only 1-2% of CD25bright cells in the normal human body have immunosuppressive function. Therefore, the lack of a sufficient number of Tregs limits the feasibility of clinical applications; 2) the phenotype and inhibitory function may be altered after amplification. Although it is possible to overcome the problem of the number of cells by amplification in vitro, however, it has recently been reported that Tregs in vitro of multiple rounds lost the immunosuppressive ability, and the polyclonal-amplified Tregs could not prevent GVHD after hematopoietic stem cell transplantation; it is also found that Tregs could be transformed into effector T cells under certain conditions; 3) once natural Tregs are widely activated by anti-CD3 monoclonal antibody or PHA, then its mediated inhibitory function is not antigen specific, that is, they can inhibit both T cells with the same antigen specificity and T cells specific for other antigens; 4) natural Treg cells can be transformed into Th17 cells under the induction of the proinflammatory cytokine IL-6. Th17 cells are a Th subpopulation of cells that produce IL-17A and IL-17F. IL-17 is a proinflammatory cytokine involved in the development and progression of many inflammatory and autoimmune diseases. In addition, Th17 cells are also closely related to rejection of the transplant.

In recent years, Tregs have been mainly amplified by magnetic beads coated with CD3/CD28. Although a large number of cells can be obtained under this amplification method, the technique also has its drawbacks: 1) the culture tends to cause a decrease in Foxp3 of cells; 2) the longer the amplification time is, the lower the Tregs function will be; and 3) the culture cost is high. Mature DC-induced antigen-specific regulatory T cells provide a single endogenous antigen and are capable of treating xenogeneic graft-versus-host disease (xGVHD). Recent studies have shown that this DC-induced antigen-specific T cells have a certain ability to expand in vivo and has an extraordinary ability to inhibit cytokine expression. Therefore, DC-induced regulatory T cells have extremely high clinical value for clinical treatment of autoimmune diseases and anti-rejection therapy after organ transplantation.

### SUMMARY OF THE INVENTION

The object of the invention is to provide a method for amplifying antigen-specific regulatory T cells in vitro in view of the above disadvantages in the prior art, and the regulatory T cells produced by this method are not only abundant in number, but also stable and difficult to be transformed, and can be used as a good immunosuppressive agent.

The invention adopts Rapamycin combined with TGF-β cells to induce human T cells into antigen-specific regulatory T cells with immunosuppressive function in vitro by the action of DC cells, making it a sufficient number of regulatory T cells for clinical research and treatment, the role of which is to activate the signal path of T cell receptors (TCR) in naive T cells sorted by human peripheral blood lymphocytes by Rapamycin in combination with TGF-β and treated DC cells in vitro, which achieves the purpose of increasing the transcription of Foxp3. Foxp3 is an existing marker for regulatory T cells, so the combination of Rapamycin and DC cells effectively promotes T cells activation and promote expansion of antigen-specific regulatory T cells in vitro.

In order to solve the technical problems above, the method of the invention comprises the following steps:

step 1, collecting: collecting blood with routine blood collection of heparin anticoagulation;

step 2, isolating: centrifugally isolating peripheral lymphocytes from the collected blood, then isolating the peripheral lymphocytes to obtain the original CD4+CD45RA+ T cells;

step 3, preparing DC cells: selecting the blood of the donor with the HLA phenotype different from that of T cells in step 2, and after being isolated by lymphocyte separation, sorting the CD14+ cells, and stimulating with GM-CSF (1000 U/ml) and IL-4 (1000 U/ml) for 6 days; DC cells are irradiated (30 Gy) before amplification;

step 4, first amplifying: stimulating the sorted CD4+CD45RA+ T cells, and adding the irradiated DC cells and IL-2, IL-15, and TGF-β to culture for 11 days; counting the number of cells every three days, and sub-culturing and supplementing the medium and cytokine according to the cell density;

step 5, second amplifying: on the 11th day, adding once more the irradiated DC cells and IL-2, IL-15, and TGF-β to re-stimulate according to the cell concentration, and culturing until the number of cells reaches the target amplification number, then collecting the cells to obtain the CD4+CD25+ regulatory T cells.

Wherein:

in step 4, the medium is prepared by adding penicillin (100 U/ml), streptomycin (100 ug/ml), 2 mM of 1-glutamic acid, 10 mM of 4-hydroxyethyl piperazine ethanesulfonic acid, 0.1 mM of non-essential amino acid, 1 mM of sodium pyruvate and 50 mM of dihydroxy ethanol to the complete RPMI-1640 medium. The stimulation method is the stimulation by T cell surface receptors, and stimulation is performed by DCs. The ratio of Tregs to DC cells is 10:1. Since DC cells are irradiated, they will die after 5 days. The stimulating agent comprises IL-2 (100 U/ml), IL-15 (10 ng/ml), TGF-β (100 ng/ml), and 10 nM of rapamycin.

In step 4 and 5, DC cells are derived from PBMCs of other donors that are incompatible with T cells HLA. The sorted DC cells are added with antigen peptides to be stimulated and matured, and the cells are irradiated on the day of induction and expansion of T cells, with the irradiation equivalent of 30 Gy. Counting the number of cells every three days during induction and amplification, and adding the medium to maintain a cell concentration of 0.5×106/ml. At the same time, adding a sufficient amount of IL-2, and adding Rapamycin in an appropriate amount to maintain the Rapamycin concentration.

The invention adopts Rapamycin combined with IL-2 to induce antigen-specific regulatory T cells into regulatory T cells with immunosuppressive function in vitro, which has the advantages as follows: 1) the amount is sufficient and can be expanded to the therapeutic amount as needed; 2) the obtained regulatory T cells can resist differentiation to Th17 cells and overcome many defects of natural regulatory T cells. In inflammatory diseases and organ transplant patients, the regulatory T cells obtained in the invention have great clinical application value.

### BRIEF DESCRIPTION OF THE DRAWINGS

The invention is further described hereinafter with reference to the accompanying drawings.

FIG. 1 is a comparison diagram illustrating the relationship between the ratio of Foxp3+ cells in the induction of T cells and antigen-specific regulatory T cells in the control group;

FIG. 2 is a comparison diagram illustrating the number of expansion of T cells and antigen-specific regulatory T cells in vitro in the control group;

FIG. 3 is a diagram illustrating the number of double positive cells of CD25 and Foxp3 in the expansion of T cells and antigen-specific regulatory T cells in the control group;

FIG. 4 is a statistical diagram illustrating the inhibitory effect of T cells and antigen-specific regulatory T cells in the control group on T cells;

FIG. 5 is a flow diagram illustrating the inhibitory effect of T cells and antigen-specific regulatory T cells in the control group on T cells;

FIG. 6 is a statistical diagram illustrating the inhibitory effect of T cells and antigen-specific regulatory T cells in the control group on LPS-stimulated T cells cytokine expression;

FIG. 7 is a flow diagram illustrating the inhibitory effect of T cells and antigen-specific regulatory T cells in the control group on LPS-stimulated T cells cytokine expression;

FIG. 8 is a diagram illustrating the survival of xenogeneic graft-versus-host disease;

FIG. 9 is a diagram illustrating the changes in body weight of xenogeneic graft-versus-host disease;

FIG. 10 is a diagram illustrating the pathological examination of xenogeneic graft-versus-host disease.

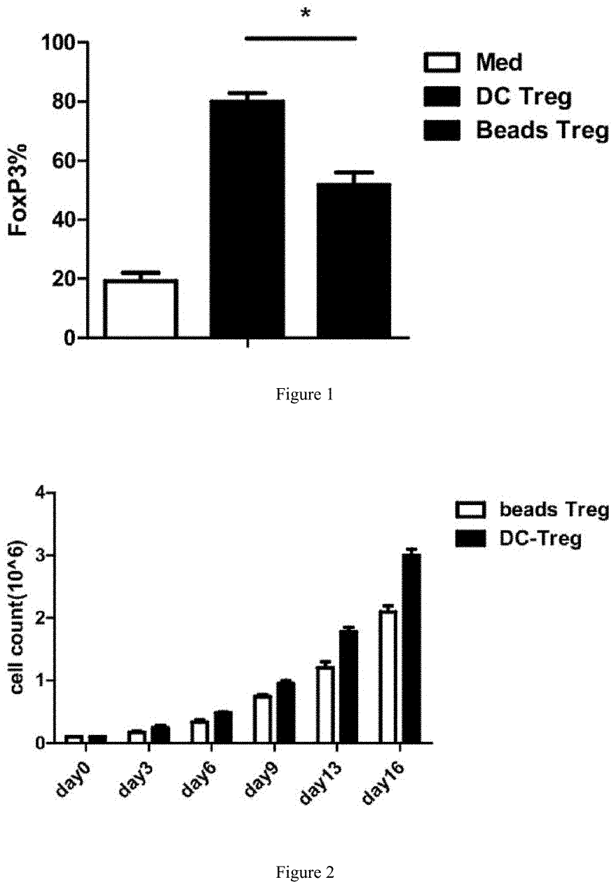

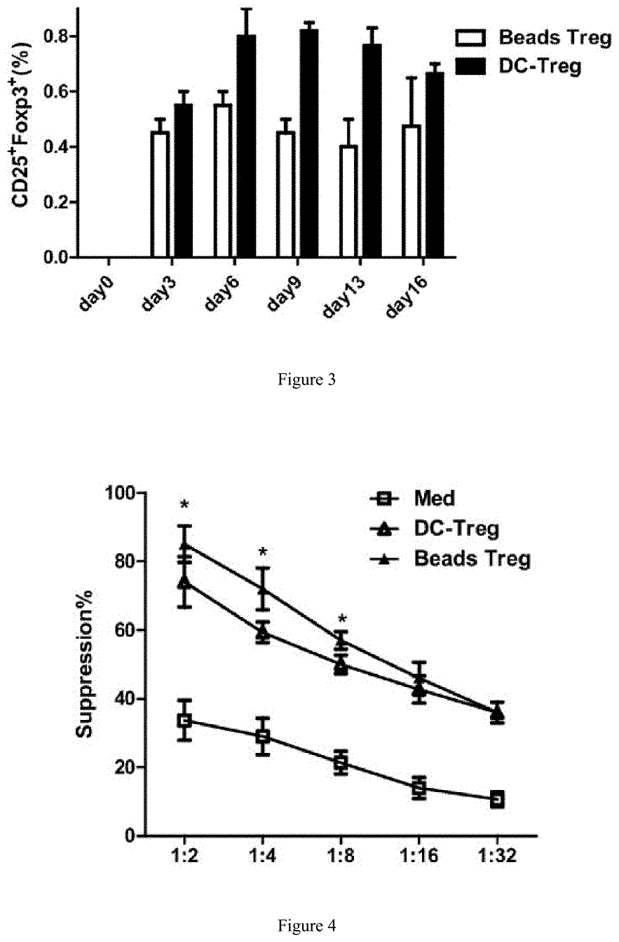

### DETAILED DESCRIPTION OF THE PREFERRED EMBODIMENTS

### Embodiment 1

Material: CD4+CD45RA+ T cells isolation kit (purchased from Miltenyi Biotec, USA), anti-human CD14 cell isolation kit (purchased from Miltenyi Biotec, USA), mice with SCID (purchased from Jackson Laboratories, USA).

Instrument: Magnetic Bead Separator (Auto MACS from Mirin Co., Germany), Flow Cytometry (BD, Model: Vantage SE)

Reagents: rhlL-2 (final concentration of 100 IU/ml after addition), rhlL-15 (10 ng/ml), and Rapamycin (final concentration of 10 nM after addition), purchased from Wyeth Pharmaceuticals. The medium is prepared by adding penicillin (100 U/ml), streptomycin (100 ug/ml), 2 mM of 1-glutamic acid, 10 mM of 4-hydroxyethyl piperazine ethanesulfonic acid, 0.1 mM of non-essential amino acid, 1 mM of sodium pyruvate (the above are purchased from BioSource International) and 50 uM of dihydroxy ethanol (purchased from Sigma Aldrich) to the complete RPMI-1640 medium.

The experimental steps are as follows:

### 1.1 Amplification of Antigen-Specific Regulatory T Cells In Vitro

Step 1, collecting: collecting blood with routine blood collection of heparin anticoagulation.

Step 2, isolating: slowly adding 20 ml of collected blood to 30 ml of human lymphocyte separation solution (purchased from Shanghai Oveida Instrument Technology Co., Ltd.), and centrifuging at 1500-2000 rpm for 25 minutes at 4° C.; pipetting the middle white cell layer after centrifugation to obtain the peripheral blood lymphocytes (PBMC), then placing the PBMC into the CD4+CD45RA+ T cells isolation kit to remove CD8, CD14, CD16, CD19, CD36, CD56, CD123, CD235 and CD45RO by negative sorting, and positively sorting the CD45RA cells in the remaining cells.

Step 3, preparing DC cells: selecting the donors with different HLA phenotypes, and isolating to obtain the peripheral blood lymphocytes (PBMC) by the same method, then placing the PBMC into the CD14 cells isolation kit, and positively sorting the DC cells; seeding the DC cells in a culture plate at a cell concentration of 0.5-1.0×106/ml, and adding GM-CSF (1000 U/ml) and IL-4 (1000 U/ml) to stimulate for 6 days.

Step 4, first amplifying: on the first day, pre-irradiating the DC cells (30 Gy) to remove the proliferative capacity and retain the cellular immunogenicity of cells; adding the irradiated DC cells to CD4+CD45RA+ T cells. The ratio of T cells to DCs is 10:1, and adding IL-2, IL-15 and Rapamycin stimulate cells proliferation; on the third day, the sixth day, and the ninth day, counting the number of cells, maintaining the cell concentration and sub-culturing; at the same time, re-adding IL-2 and adding Rapamycin in an appropriate amount.

Step 5, second amplifying: collecting the cells on the 11th day of cell culture, re-seeding into a larger cell culture flask, adding the irradiated DC cells, IL-2, IL-15 and Rapamycin that are consistent with the first stimulation concentration; after that, counting the number of cells every three days and sub-culturing, and supplementing the stimulating agents until the target number of regulatory T cells is obtained; before using the cells, performing rest on the cells in RPMI 1640 medium containing 10% FBS and rh-IL-2 (1000 IU/ml) for 48 hours, and collecting the above cells as regulatory T cells.

### 1.2 Analysis and Identification of Amplification of Antigen-Specific Regulatory T Cells In Vitro

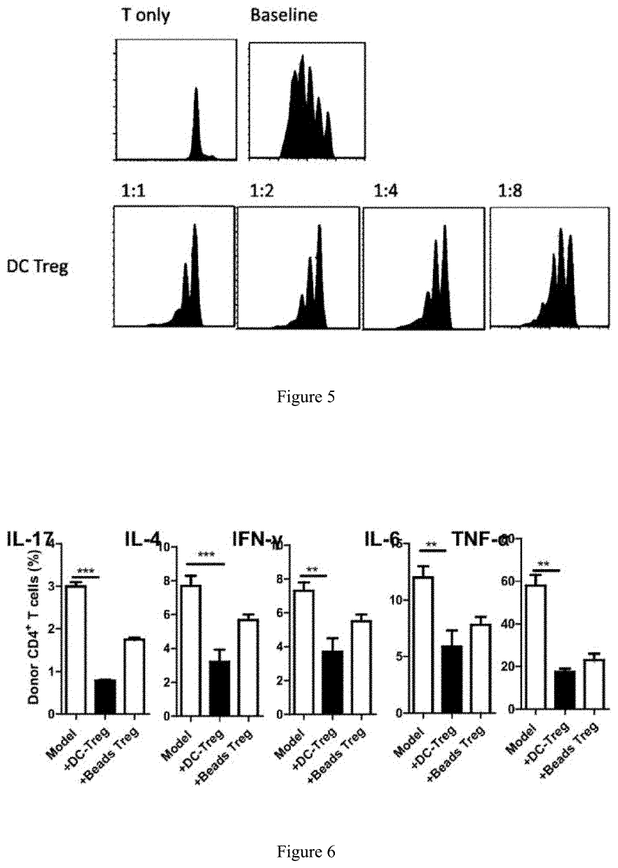

1.2.1 Coating the regulatory T cells obtained in the invention with the regulatory T cells induced by the CD3/CD28 magnetic beads to be co-cultures with CFSE fluorescently labeled T cells at a ratio of 1:5, 1:10, 1:20, and the result are shown in FIGS. 4 and 5. Antigen-specific T cells show an ability to inhibit T cells proliferation beyond regulatory T cells induced by other methods, and only the antigen-specific regulatory T cells at the ratio of 1:20 have a cytostatic function. The conclusion is that antigen-specific T cells have stronger immunomodulatory ability than magnetic bead-induced inducible regulatory T cells (iTreg).

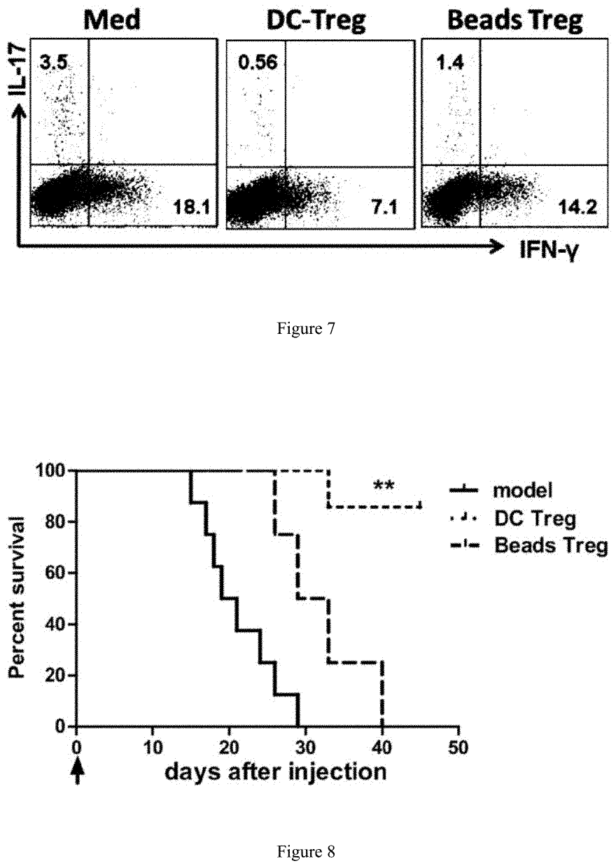

1.2.2 Co-culturing the regulatory T cells obtained in the invention, the regulatory T cells induced by anti-CD3/CD28 magnetic beads and the LPS-stimulated peripheral blood lymphocytes for three days, and detecting the cytokines by flow, the results are shown in FIGS. 6 and 7. PBMC substantially inhibits the differentiation into Th1 and Th17 cells under the action of antigen-specific regulatory T cells. The conclusion is that antigen-specific regulatory T cells have a stronger inhibitory function of inflammatory factors than magnetic bead-induced inducible regulatory T cells (iTreg).

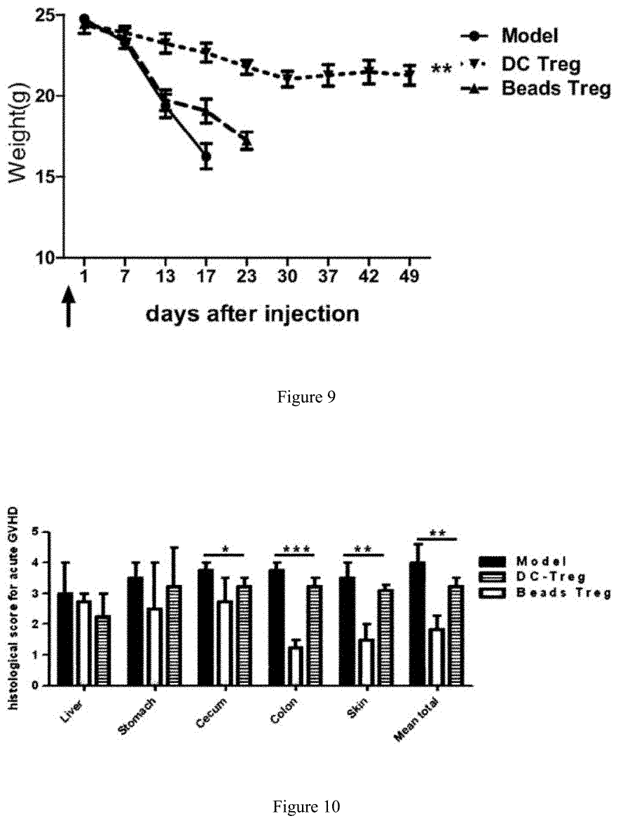

1.2.3 Injecting the antigen-specific regulatory T cells amplified by the above method alone or in combination with the allogeneic fluorescent fuel CFSE-labeled T cells into the SCID mice through the tail vein, and the regulatory T cells obtained by the method are not pathogenic by themselves, and can inhibit the proliferation of effector T cells, delay or prevent the occurrence of GVHD. As is shown in FIGS. 8 and 9, in the control group, obvious cirrhotic lobule appears in the liver, renal tubular necrosis and inflammation appears in the kidney, and fibrosis appears in the lung. The above pathological changes do not appear in the antigen-specific regulatory T cells treatment group. It can be seen that antigen-specific regulatory T cells have stronger inhibitory functions in vivo and have no toxic side effects than other in vitro induced regulatory T cells.

### 1.3 Clinical Application Examples

Taking healthy volunteers as an example, using antigen-specific regulatory T cells for the detection of toxic side effects: collecting the peripheral venous blood of five healthy volunteers and preparing according to the method in 1.1, and calculating according to the body surface area of 20×106/m2, once a month; observing the survival cycle and toxic side effects of the cells in vivo. Regulatory T cells after injection can survive in the body for 30-50 days, and do not cause adverse reactions such as fever and allergies.

The advantages of the invention from the above experiments are as follows:

(1) Antigen-specific regulatory T cells can significantly inhibit proliferation of T cells;

(2) Antigen-specific regulatory T cells have no obvious toxic side effects and have long-term protective ability in vivo;

(3) Antigen-specific regulatory T cells can replace immunosuppressive agents or reduce the dose of immunosuppressive agents, and can be used for immunotherapy in the late stage of organ transplantation and can also be used for the treatment of patients with autoimmune diseases or type I diabetes; it will be a milestone in the history of organ transplantation immunotherapy.

The invention may have other embodiments other than the embodiments above. Any technical solution formed by equivalent replacements or equivalent transformations shall all fall within the protection scope of the invention.

1. A method for amplifying antigen-specific regulatory T cells in vitro, comprising the following steps:
step 1, collecting: collecting blood with routine blood collection of heparin anticoagulation;
step 2, isolating: centrifugally isolating peripheral lymphocytes from the collected blood, then isolating the peripheral lymphocytes to obtain the original CD4+CD45RA+ T cells;
step 3, preparing DC cells: selecting the blood of the donor with the HLA phenotype different from that of T cells in step 2, and after being isolated by lymphocyte separation, sorting the CD14+ cells, and stimulating with GM-CSF (1000 U/ml) and IL-4 (1000 U/ml) for 6 days; DC cells are irradiated (30 Gy) before amplification;
step 4, first amplifying: stimulating the sorted CD4+CD45RA+ T cells, and adding the irradiated DC cells and IL-2, IL-15, and TGF-β to culture for 11 days; counting the number of cells every three days, and sub-culturing and supplementing the medium according to the cell density;
step 5, second amplifying: on the 11th day, adding once more the irradiated DC cells and IL-2, IL-15, and TGF-β to re-stimulate according to the cell concentration, and culturing until the number of cells reaches the target amplification number, then collecting the cells to obtain the CD4+CD25+ regulatory T cells.

2. The method for amplifying regulatory T cells in vitro according to claim 1, wherein in step 3, labeling the flow sorted DC cells with CD14, and pretreating the DC cells with GM-CSF and IL-4.

3. The method for amplifying regulatory T cells in vitro according to claim 1, wherein in step 4, activating T cells with the irradiated DC cells.

4. The method for amplifying regulatory T cells in vitro according to claim 1, wherein in step 5, on the 11th day, re-stimulating the regulatory T cells with the irradiated DC cells, IL-2 and IL-15, to promote the re-activation and re-amplification of T-cells
